(19) United States
(12) Plant Patent Application Publication
(10) Pub. No.: US 2006/0236437 P1
(43) Pub. Date: Oct. 19, 2006

(54) Strawberry variety named 'Wendy'

(76) Inventor: Andrew Raymond Jamieson, Kentville (CA)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 11/274,388

(22) Filed: Nov. 15, 2005

(30) Foreign Application Priority Data
Apr. 18, 2005 (CA) 05-4723

(51) Int. Cl. A01H 5/00

(52) U.S. Cl. PLT/208

(57) ABSTRACT

This invention relates to a new and distinct variety of strawberry (Fragaria xananassa) named ‘Wendy’. The invention is an early season, short-day variety similar to ‘Evangeline’. ‘Wendy’ is distinguished from ‘Evangeline’ by its flower characteristics, calyx characteristics, fruit size, fruit shape, and insertion of the achenes.

### CROSS-REFERENCE TO RELATED APPLICATIONS

This application claims priority under 35 U.S.C. § 119 to Plant Breeder's Rights Certificate Application No. 05-4723, filed in Canada on Apr. 18, 2005, the disclosure of which is incorporated herein by reference in its entirety.

### BOTANICAL CLASSIFICATION

The present invention relates to a new Fragaria plant.

### VARIETY DENOMINATION

The new plant has the varietal denomination ‘Wendy’.

### BACKGROUND OF THE INVENTION

The present invention includes a new and distinct cultivar of Fragaria xananassa known by the varietal name ‘Wendy’, originally designated as ‘K98-6”. The new variety resulted from a controlled cross in an ongoing breeding program between the strawberry plants ‘K96-5’ and ‘Evangeline’. ‘K96-5’ is an unpatented variety developed by the Atlantic Food and Horticulture Research Centre (a facility of Agriculture and Agri-Food Canada) in Kentville. ‘Evangeline’ is a variety developed by Andrew R. Jamieson for which Canadian Plant Breeder's Rights were granted in 1999 (No. 06170). ‘Wendy’ was discovered in 1998 as a seedling in a controlled breeding plot near Sheffield Mills, Nova Scotia at the Sheffield Farm, a field-station of the Atlantic Food and Horticulture Research Centre, where it was selected and propagated asexually by stolons at the Atlantic Food and Horticulture Research Centre in Kentville. Asexual propagules from this original source have been produced annually in a greenhouse at the Atlantic Food and Horticulture Research Centre, Kentville, Canada. ‘Wendy’ has been tested at the Atlantic Food and Horticulture Research Centre (starting in 1999) and also at the Agriculture and Agri-Food Canada research centre at Buctouche, New Brunswick, both of Canada. This propagation and testing has demonstrated that the combination of traits disclosed herein which characterize the new variety are fixed and retained true to type through successive generations of asexual reproduction via stolons.

### BRIEF SUMMARY OF THE INVENTION

The present invention relates to a new and distinct variety named ‘Wendy’. The variety is botanically identified as Fragaria xananassa. The variety is typical of short-day varieties and produces fruit over a four week period in northern temperate climates. The new variety is distinguished from other varieties by a number of characteristics as set forth in Tables 1,2, and 3.

### BRIEF DESCRIPTION OF THE DRAWINGS

The accompanying photographs show typical specimens of the new variety including fruit, foliage and flowers, in color as nearly true as it is reasonably possible to make in color illustrations of these characteristics.

FIG. 1 shows plant parts of the new variety, typical in size, shape, and color.

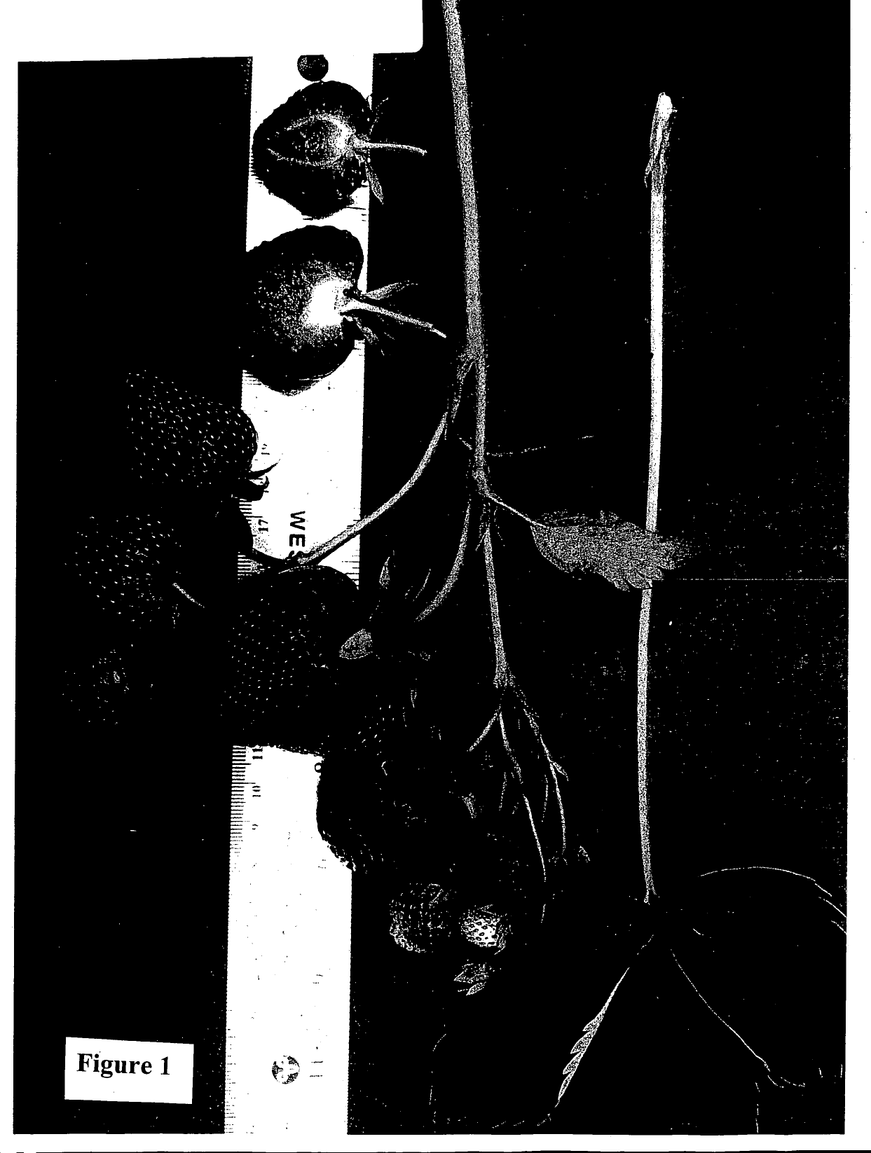

FIG. 2 shows the flowers of the new variety (on the left) compared with Evangeline (on the right).

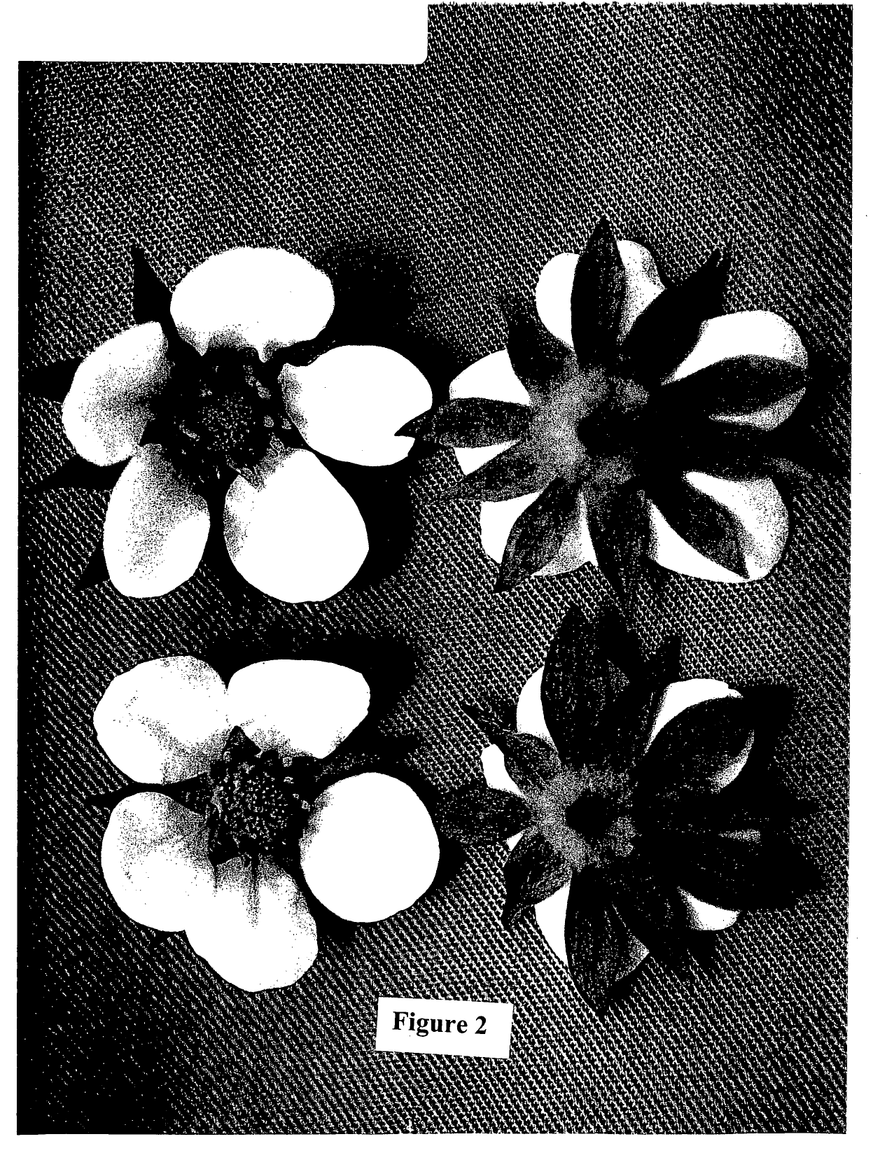

FIG. 3 shows primary fruit of the new variety.

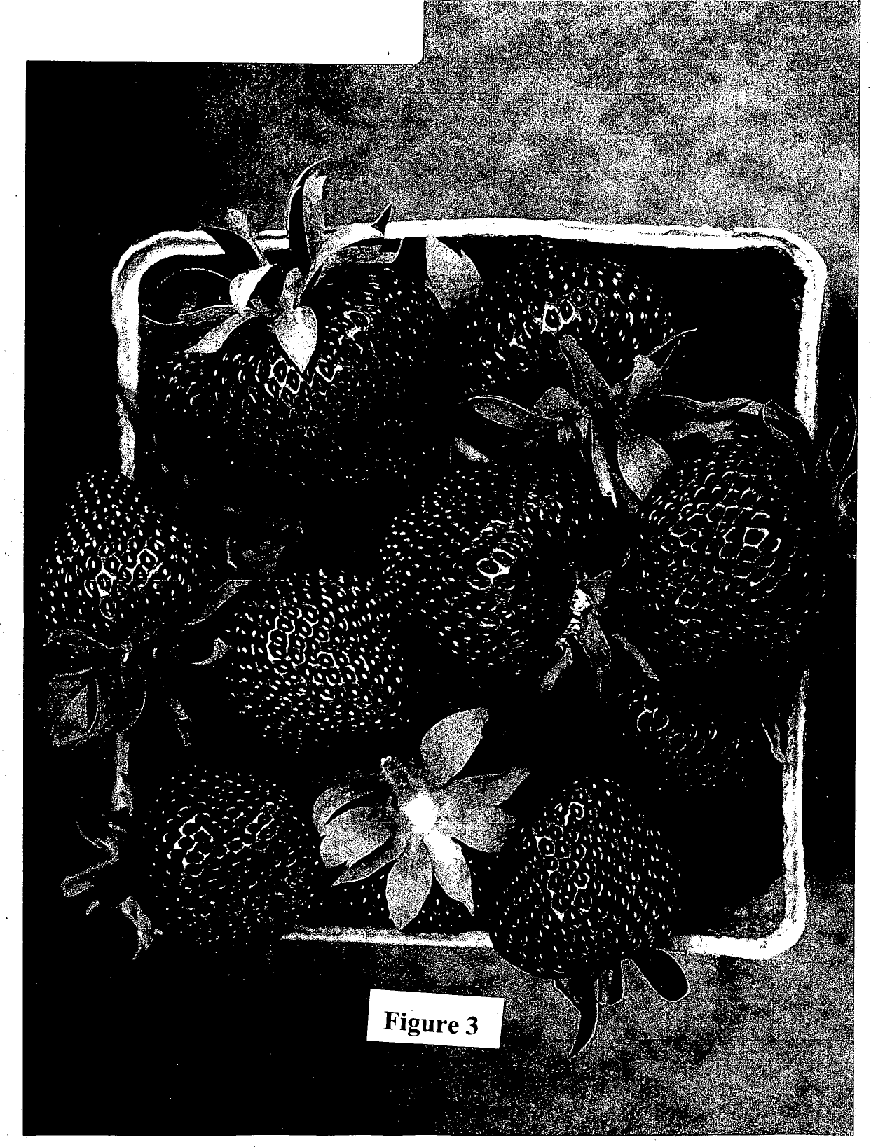

### DETAILED BOTANICAL DESCRIPTION

The following detailed botanical description of the new variety, and the similar variety ‘Evangeline’, is based upon measurements and observations taken of plants and fruit grown in Kentville, Nova Scotia, Canada. Observations were taken from both varieties as grown hi a side-by-side field trial in 2002, 2003, 2004 and 2005. This description is in accordance with UPOV terminology. Color designations, color descriptions, and other phenotypical descriptions may deviate from the stated values and description depending upon variation in the environment, seasonal, climatic and cultural conditions, however, it is believed that this description will apply to the ‘Wendy’ plants grown in similar conditions of soil and climate elsewhere. Colors are described using a standard R.H.S. Color Chart. Descriptive information on the new variety is presented in Tables 1 and 2 in the tables, the flowers described are secondary flowers. The fruit described is the secondary fruit of the maiden crop, thirteen or fourteen months after planting. The harvest data in Table 3 is based on a complete harvest of the crop. Principal differences between ‘Wendy’ and the variety ‘Evangeline’ are set forth.

The new variety is botanically identified as Fragaria xananassa and commercially classified as a short-day strawberry.

Plant and foliage characteristics: When propagated in the nursery, ‘Wendy’ produces stolons freely; producing similar numbers of daughter plants as ‘Evangeline’. Individual plants of ‘Wendy’ are medium to large. The plants of ‘Wendy’ are of medium density with globose habit and strong vigor. As shown in Table 1, leaf color of ‘Wendy’ and ‘Evangeline’ are Green Group 137A on the upper surface and lighter Green Group 137C on the under surface. Leaflets of ‘Wendy’ and ‘Evangeline’ are about the same size, shape and convexity, and leaves of both varieties have three leaflets. The leaflet serrations of ‘Wendy’ are similar in size to ‘Evangeline’ but for ‘Wendy’ serrations are more rounded and less pointed than for ‘Evangeline’ and they have a few more serrations than for ‘Evangeline’ and, as a result, the portion of the leaf margin without serrations is smaller. The venation of ‘Wendy’ leaflets is pinnate. Petiole pubescence for ‘Wendy’ tends to be perpendicular to the petiole, whereas petiole pubescence for ‘Evangeline’ tends to be oriented upwards, especially near the point of attachment of the leaflets.

TABLE 1
Foliar characteristics for ‘Wendy’ and ‘Evangeline’.

Cultivar

| Foliar Character | ‘Wendy’ | ‘Evangeline’ |
|---|---|---|
| Leaf color | Green Group | Green Group |
| Upper surface | 137A | 137A |
| Lower Surface | 137C | 137C |
| Central leaflet | | |
| Length (mm) | | |
| Mean | 92.8 | 99.1 |
| Range | 83-99 | 83-114 |
| Width (mm) | | |
| Mean | 78.2 | 79.2 |
| Range | 68-87 | 65-93 |
| Length/width ratio | 1.19 | 1.25 |
| No. leaflets/leaf | 3 | 3 |
| Leaf convexity | cupped | cupped |
| Serrations | | |
| Number | | |
| Mean | 21.0 | 18.2 |
| Range | 19-22 | 16-20 |
| Size | medium | medium |
| Shape | semi-round | semi-pointed |
| Tip serration size | small | small |
| Leaf pubescence | medium | medium |
| Petiole pubescence | | |
| Density | medium | sparse-medium |
| Direction | perpendicular | perpendicular to upwards |

Flower and fruit characteristics: The length of bloom for ‘Wendy’ is about three weeks when grown in Kentville, Nova Scotia, Canada in a matted row cultural system. Flowering for ‘Wendy’ and ‘Evangeline’ typically begins on May 21 and ends on June 11. As shown in Table 2, the flower truss of ‘Wendy’ averages 3.7 cm longer than for ‘Evangeline’ but flowers of both varieties open even with the leaf canopy or slightly below. ‘Wendy’ flower trusses produce slightly more flowers than ‘Evangeline’. Flowers of ‘Wendy’ and ‘Evangeline’ are white which is not included on the R.H.S. Color Chart. The anther color is Yellow-Orange Group 17 A. Secondary flowers of ‘Wendy’ usually have 5 petals, occasionally 6, and ‘Wendy’ flowers are smaller than those of ‘Evangeline’. Petals of both varieties are longer than wide. Petals of ‘Wendy’ generally touch while those of ‘Evangeline’ are spaced. Trusses of ‘Wendy’ are semi-erect at first picking while those of ‘Evangeline’ are erect. The outer calyx of ‘Wendy’ is larger than for ‘Evangeline’ but the inner calyx is about the same size. The position of the calyx is even with the top of the fruit for ‘Wendy’ but raised for ‘Evangeline’. The calyx is easily separated from the fruit for both varieties. The secondary fruit of ‘Wendy’ are larger than for ‘Evangeline’ and conic in shape while the fruit of ‘Evangeline’ are conic to long conic, and generally longer than wide. The seeds of ‘Wendy’ are even with the surface of the fruit or slightly indented whereas ‘Evangeline’ seeds are more deeply indented. The flesh of both varieties is considered firm. The fruit color of ‘Wendy’ varies between the lighter Red Group 45A to the darker Red Group 46A. The pith of ‘Wendy’ is similar to or a bit darker than the pith of ‘Evangeline’ but the cortex is the same color. The achenes of ‘Wendy’ are Green-Yellow Group 1A but darken to Orange-Red Group 34 A on the side of the fruit when exposed to sunlight.

TABLE 2
Flower and fruit characteristics for ‘Wendy’ and ‘Evangeline’

| | Cultivar | |
|---|---|---|
| Character | ‘Wendy’ | ‘Evangeline’ |
| Flower Position | even or | even or |
| (relative to leaf canopy) | slightly beneath | slightly beneath |
| | medium | short-medium |
| Flower truss length | 26.8 | 23.1 cm |
| Number of flowers/truss | 9.0 | 8.3 |
| Number of petals | 5.2 | 5.0 |
| Flower size (mm diameter | 32.4 | 35.8 |
| Petal length (mm) | 13.7 | 15.3 |
| Petal width (mm) | 12.2 | 12.6 |
| Petal spacing | touching | spaced |
| Calyx size | | |
| Inner calyx (mm diam.) | 32.3 | 32.1 |
| Outer calyx (mm diam.) | 36.8 | 33.4 |
| Calyx position | even | raised |
| Adherence of the calyx | weak | weak |
| Fruit size and shape | | |
| Length (mm) | 32.2 | 31.8 |
| Width (mm) | 32.8 | 30.3 |
| Length/width ratio | 0.98 | 1.05 |
| Subjective fruit shape | conic | conic to long-conic |
| Achene position | even to | indent |
| | Slightly indented | |
| Fruit firmness | firm | firm |
| Fruit firmness (Newtons) | 4.9 | 4.5 |
| Color (R.H.S. Color Chart) | | |
| Calyx | Green Group | Green Group |
| | 141C to 143A | near 141C |
| Fruit exterior | Red Group | Red Group |
| | 45A To 46A | 45A to 46A |
| Fruit interior | Red Group | Red Group |
| Pith | 45A to 46B | 45A |
| Cortex | 45A to 46B | 45A to 45B |

Disease resistance: Wendy’ and ‘Evangeline’ are susceptible to red stele root rot (Phytophthorafragariae). ‘Evangeline’ is resistant to powdery mildew (Sphaerotheca macularis) while ‘Wendy’ is moderately resistant. ‘Wendy’ and ‘Evangeline’ are moderately resistant to fruit rot (Botrytis cinerea).

Production characteristics: ‘Wendy’ has been widely tested for several years and in replicated trials planted in 2003 and harvested in 2004. ‘Wendy’ has consistently produce higher yields than ‘Evangeline’, as shown in Table 3. The percent marketability of both varieties is similar, indicating the lack of problems with pollination or fruit diseases. The fruit of ‘Wendy’ are much larger (by weight) than ‘Evangeline’. The mean harvest date for ‘Wendy’ is one day later than for ‘Evangeline’. Subjectively, the flavor of the fruit of ‘Wendy’ is rated very good to excellent when fully ripe.

TABLE 3
Performance of ‘Wendy’ and ‘Evangeline’ for 2004 at Kentville,
N.S. and Bouctouche, N.B. Plants were grown in matted rows and
three blocks of 3 m long rows were harvested at each site.

| | Total yield | % yield | Size | Mean day |
|---|---|---|---|---|
| | (t/ha) | marketable | (g/fruit) | of harvest |
| 2004 (Kentville) | | | | |
| ‘Wendy’ | 21.1 | 90.3 | 17.1 | July 10 |
| ‘Evangeline’ | 10.8 | 88.5 | 11.1 | July 9 |
| 2004 (Bouctouche) | | | | |
| ‘Wendy’ | 8.3 | 85.0 | 13.3 | July 13 |
| ‘Evangeline’ | 5.8 | 90.1 | 9.9 | July 12 |

What is claimed:

1. The new and distinct variety of strawberry plant named ‘Wendy’ as described and illustrated herein.
